(19) United States
(12) Patent Application Publication
(10) Pub. No.: US 2003/0135214 A1
(43) Pub. Date: Jul. 17, 2003

(54) System, device, composition and method for treating and preventing avascular or osteonecrosis

(76) Inventors: Joseph F. Fetto, New York, NY (US); Alejandro Leali, Hackensack, NJ (US)

Correspondence Address: Joseph F. Fetto, M.D.,, Alejandro Leali, M.D., Suite 7-J, 300 E. 56th St., New York, NY, 10022, US

(21) Appl. No.: 10/050,337

(22) Filed: Jan. 15, 2002

(51) Int. Cl. A61B 17/56

(52) U.S. Cl. 606/72; 606/76

(57) ABSTRACT

A vascular necrosis of the hip is a common disease that usually affects a young, active patient population. As the disease progresses the undermined structural integrity of the subchondral bone leads to articular collapse and subsequent osteoarthrosis. The ideal treatment is one that hinders or arrests the progression of the disease averting articular collapse and a joint replacement surgery. Several non-surgical and surgical procedures have been described to treat avascular necrosis: core decompression, osteotomies of the hip, non-vascularized and vascularized bone grafts. The purpose of this paper is to describe a surgical strategy that attempts to address the multiple factors involved in the progression of the disease: deficits in structural support, increased intraosseous pressure and the bone healing process. This is accomplished through a routine core decompression procedure combined with the insertion of two bone interference screws into the subchondral bone of the femoral head to provide structural support and the use of osteoinductive bone allograft (demineralized bone matrix) in an effort to accelerate the bone response. Prospective studies are currently underway to assess long-term outcomes.

### FIELD OF THE INVENTION

This invention is concerned with the field of treatment and prevention of pain, discomfort, deformity or disability associated with loss of bone structure, strength or support that accompanies osteonecrosis or avascular necrosis of various load-bearing musculoskeletal structures.

### BACKGROUND OF THE INVENTION

Osteonecrosis (avascular necrosis, aseptic necrosis or subchondral avascular necrosis) does not represent a particular disease but the final common pathway of a number of conditions leading to bone death. Trauma, corticosteroids, alcoholism and connective tissue disorders have been historically linked to the occurrence of avascular necrosis. It most commonly involves the femoral head, followed by the knee, the humeral head, and the small bones of the wrist and foot.

The prevalence of avascular necrosis is unknown; however, 10,000 to 20,000 new cases of avascular necrosis of the hip have been estimated to develop every year [1]. Approximately 10 to 12 percent of the total hip arthroplasties performed in the U.S. are a consequence of avascular necrosis and the ensuing articular collapse and osteoarthrosis [2]. The average age of patients undergoing total hip replacement for avascular necrosis remains below 40 years [3] with a life expectancy exceeding forty years [4].

Histologically, avascular necrosis is characterized by areas of necrotic bone marrow and trabeculae extending to the subchondral plate. The overlying articular cartilage remains viable as it receives nutrition from the synovial fluid. Shortly after the initial injury, an acute inflammatory reaction is triggered and vascular fibrous tissue is deposited [5-7]. During this period, the resorption and removal of the unviable tissue occurs along with the deposition of new immature woven bone. Radiographically, the combination of irregular areas of bone deposition and bone resorption can be appreciated as ‘fragmentation’ or areas of sclerosis interspersed with areas of increased density [8]. Gross subchondral mechanical failure leads to collapse and subsequent osteoarthrosis during the interval between the removal of necrotic bone and the calcification of vascularized mature bone. Excessive stresses during this ‘vulnerable gap’ lead to articular collapse. Frequently, a radiolucent line (crescent sign) can be identified beneath the subchondral bone of the superior portion of the femoral head in the anteroposterior or lateral view representing the collapse of dead cancellous bone that separates from the articular cartilage. The antero-lateral margin of the acetabulum usually creates an indentation in the unsupported articular cartilage which compacts the weakened underlying subchondral bone [9].

After structural failure of the subchondral bone, most patients will eventually require a total hip replacement [10]. Since avascular necrosis affects relatively young patients, the problem is further compounded by their long life expectancy, high activity and demand on the implants [4].

Avascular necrosis of the femoral head is a relentless process that usually leads to articular collapse and significant disability in a young, active population. Ohzono et al. reported progression to collapse in 68% of 115 patients treated conservatively. [11] Mont et al. reviewed forty two reports encompassing 2025 patients from the published literature comparing non-operative management and core decompression and found satisfactory clinical results in only 22.7% of the patients treated conservatively and 63.5% of those treated surgically [12]. Even though the outcome of cementless total hip replacements in this group of patients seems to be encouraging [13-15], they will likely require more than one procedure throughout their lifetime [16, 17].

Although core decompression was initially described as a diagnostic and investigative tool [18] its therapeutic value was readily recognized. [19] The main benefits of the method are low morbidity, simple technique and early promising results. Core decompression is supposed to relieve intraosseous pressure [20] due to interstitial edema improving vascularity [21] and promoting bone healing. In addition, the decrease in intraosseous pressure results in manifest pain relief. The effectiveness of the procedure varies widely. The rate of progression to a total hip replacement ranges from 31% [22] to 57%. [23] The preoperative stage of the disease [24, 25], the extent and location of the lesion [26, 27], and history of corticosteroids or alcohol intake [28, 29] accounts for the differences in survival rates and clinical success associated with core decompression.

Cortical bone grafts have been used in the treatment of osteonecrosis to increase structural support to the articular cartilage after core decompression. Cortical strut allografts are usually harvested from the pelvis, fibula or tibia and inserted into the drilled canal in the femoral head [30]. The published long-term clinical results with bone grafting after core decompression are equally variable. Clinical success has been reported to range from 29% to 90% [31] [32, 33] [34] depending on the length of the follow-up and the preoperative stage of the necrotic lesion.

The erratic incorporation of the non-vascularized allografts into the host bone led to the use of vascularized bone grafts. Vascularized bone grafts have shown to incorporate more rapidly and predictably than the non-vascularized counterparts. In addition to the structural support, vascularized bone grafts introduce a source of mesenchymal stem cells and a well defined vascular supply [35] [36] [37]. Furthermore, the vascularized periosteum of the graft seems to improve the blood supply and helps initiate the revascularization and osteogenesis of the femoral head [38]. The reported clinical results appear to be more favorable when compared with core decompression [39] being an effective procedure for the precollapse stages and a valuable alternative for patients with advanced stages of the disease [40]. The technique however, is not devoid of several disadvantages. The most important are the increased morbidity associated with the donor site [41] and the femoral neck itself [42] as well as the prolonged postoperative rehabilitation phase. Additionally, it requires considerable technical expertise, the participation of two operating teams and a prolonged surgical time.

In U.S. Pat. No. 5,755,809, there was disclosed a method of femoral head core channel filling with a prosthesis. However, the prosthesis was not disclosed to be an allograft or xenograft bone implant, and there is no disclosure or suggestion of using an osteoinductive substance in combination with the disclosed prosthesis. Accordingly, there remains a need in the art for an improved method of treating and preventing avascular necrosis.

The ideal goal of any ‘early’ treatment is to delay or arrest the progression of the disease before the articular collapse and the subsequent total hip arthroplasty. Since the disease progresses as a consequence of the underlying mechanical failure, which in turn results from an impaired attempt to heal the necrotic subchondral gap, most clinicians agree that the best time to intervene is early in the history of the disease before the collapse of the femoral head (Ficat Stages I, Ia and IIb).

The approach presented herein addresses the processes involved in the progression of the disease. The elevated intraosseous pressure is dealt with by means of a routine core decompression procedure. Bone healing is enhanced with the introduction of osteoinductive material (demineralized bone matrix, growth factors, angiogenic factors and combinations thereof) inside the reamed femoral canal and finally the structural deficit is addressed with the insertion of at least one but preferably two support structures, preferably screws made from bone or another biologically compatible, preferably bioresorbable material.

### SUMMARY OF THE INVENTION

This invention provides a system, devices, compositions and methods for treating and preventing avascular necrosis at a number of biological sites in the musculoskeletal system. The system consists of creating one or more channels to provide access to the necrotic site, implantation of compositions which conduct or induce the formation of new bone tissue at the site, and implantation of biologically compatible support structures adjacent the site to prevent collapse of the necrotic tissue pending formation of new, vascularized bone tissue at the site which replaces the necrotic tissue.

Accordingly, it is the purpose of this invention to provide a method for the prevention and/or reduction of the complications associated with osteonecrosis and its treatment.

It is a further object of this invention to provide a kit comprising at least one surgical drill, means for delivery of a composition, as well as the composition itself, which induces the formation of new bone at the site of necrosis, and biologically compatible support means pending formation of new bone.

It is a further object of this invention to provide a surgical method whereby the complications associated with osteonecrosis and its treatment prevented and/or reduced.

Other objects and benefits of this invention are apparent from a review of the complete disclosure and the included claims.

### BRIEF DESCRIPTION OF THE DRAWINGS

FIG. 1. Preoperative magnetic resonance image. Right: Axial view. The affected area of the femoral head has been delineated. Left: Saggital view.

FIG. 2. Surgical technique. Left: Model demonstration. Introduction of bone screw after proper reaming and final tapping, into subchondral bone in the affected area. Right: Intraoperative view of allograft bone screw placement under image intensification.

FIG. 3. Surgical technique. Left: Lateral view of guide wire positioning in the femoral head. Right: Lateral view of final tapping into the subchondral plate in the second reamed canal. Note that both diverging paths were drilled through a single entry hole.

FIG. 4. Radiographic A-P views. Left: Preoperative film. Note the irregularity of the articular surface in the pre-collapse stage. Right: Postoperative film showing the outline of the bone screws (arrows).

FIG. 5. 4 week Follow-up films: Left: A-P Projection. Right: Lateral projection. Note the diverging paths of the screws from a single entry hole to provide bi-plane support of the lesion. There is evidence of newly induced bone formation around the screws and inside the reamed canal.

FIG. 6. Example of cortical bone screw for use according to this invention.

### DETAILED DESCRIPTION OF THE PREFERRED EMBODIMENTS OF THE INVENTION

Those skilled in the art will appreciate that a wide variety of techniques have been applied to the attempted treatment and prevention of avascular necrosis. Unfortunately, to date, no system has been devised which adequately addresses this condition. Mere core decompression is inadequate, and often results in the need for total hip replacement, when that is the site of the osteonecrosis. It will further be understood that avascular necrosis may occur at a number of articulating sites where load is carried. Thus, the hip, knee, shoulder, foot, hand, and more specifically, the femur, in the femoral or tibial condyles or epicondyles, the malleolus of the tibia or fibula, the head or tubercles of the humerus, or either terminus of the radius or ulna, talus, carpal navicular (for hand surgery, i.e. modification of the known “Bennett Screw”), and other locations are all susceptible to this debilitating and painful condition. The present invention provides a system whereby the vast majority of the most serious forms of avascular necrosis may be managed, treated, or prevented in each of these sites.

The essential features of this invention include the creation of a means of access to the necrotic site, implantation therein of a composition comprising bone morphogenetic protein, growth factors, angiogenic factors, and optionally, additional biologically active compositions, such as antibiotic compositions, which would assist in the resolution of any infection that may have developed at the site of necrosis. The implanted composition should ideally include osteoinductive elements, such as bone morphogenetic protein, BMP, natural or recombinant, or a more complex source of such osteoinductive elements, such as demineralized bone, from which such osteoinductive elements as BMP may diffuse into the necrotic site. Osteoconductive substances, such as bioactive glass, or other biologically active ceramic material, calcium phosphate, or other calcium or phosphate salts, bone chips, including cortical bone, cancellous bone, or mixtures thereof, are also desirable components of a composition to be implanted within or adjacent to the necrotic site. Such components provide added load bearing capacity while the lengthier process of new bone formation and remodeling of implanted substances continues. Finally, in order to maximize the opportunity for repair and recovery, and to retain any implanted compositions at the site of the necrosis, one or more load supporting devices are implanted adjacent the necrotic site in a manner to optimally support any load that might be experienced at the necrotic site, pending formation of new bone at the necrotic site.

Generally, patients present with hip pain, or pain at the knee, only after substantial necrosis has developed at the site. Frequently, a patient that has undergone total hip replacement, for example, should be checked on the contralateral side, to determine whether the same or similar necrotic process may not have set in. If evidence of necrosis is found, either by X-ray, NMR or other diagnostic technique known in the art to be helpful in identifying the biological sites in the musculoskeletal system afflicted by osteonecrosis, then the method and system of the present invention may be employed to prevent the need for a further total hip or knee replacement procedure. As this methodology becomes more generally accepted, it is hoped that as part of a routine physical checkup, the need for this methodology to be implemented for a given patient will be appreciated and the invention utilized in a prophylactic manner. Accordingly, identifying the need for this treatment is a critical component to the successful implementation of this invention.

Once the need for this treatment has been confirmed, the method is practiced by creating at least one channel from the exterior of the biological site in the musculoskeletal tissue into or proximal the necrotic site, so that access to the necrotic site is facilitated. In the case of necrosis in the femoral head, this is achieved by drilling a single hole into the lateral cortex of the proximal femur, and defining preferably two channels from the single hole that is created. The two channels should be directed in a “V” shape, with the vertex of the “V” being at the single hole, and the two arms of the “V” extending toward and below the site of necrosis, through the femoral neck into the femoral head. Care must be taken not to excessively weaken the femoral neck by drilling channels of too great a diameter. In addition, the diameter of each channel should be maintained at between about 5 to about 10 mm. Preferably, the channel diameter will be maintained at between about 6 mm and about 8 mm. In the case of AVN of the femoral head, each channel may need to extend into the femoral head, up to the level of the subchondral surface where necrosis generally develops.

Once the channel(s) is/are created, a composition is inserted therein which will, in time, induce the formation of new bone, while at the same time, preferably providing support to the subchondral surface. Preferred compositions for this purpose include, but are not limited to OSTEOFIL® Paste Products, REGENAFIL® Paste Products, REGENAFORM™ Moldable Blocks, OPTEFORM™ Paste, REGENAPACK™ Paste Squares, OSTEOPACK™ Paste Squares, all of which are available from Regeneration Technologies, Inc. These compositions are described in detail in published PCT application number PCT/US98/04904, WO98/40113 (published Sep. 17, 1998), herein incorporated by reference for this purpose. Essentially, these compositions comprise an inert biological carrier of gelatin, an osteoinductive substance including demineralized bone matrix (DBM), and in some cases cortical bone chips, cancellous bone chips, or both. Other substances that may be utilized to advantage in this procedure include a composition comprising DBM in a glycerol carrier or a composition comprising DBM in a alginate carrier as described in U.S. Provisional Application No. 60/343,943 (incorporated herein by this reference). Bioactive glass, and various salts of calcium and phosphate may also be used to advantage for this purpose. Accordingly, any substance known to have bone inducing properties, or the ability to conduct the formation of new bone, are useful in this regard, with materials that induce new bone formation being preferred. In addition, there are many angiogenic materials known in the art. Inclusion of angiogenic materials in the composition is of assistance to ensure that any new bone that is formed is adequately supplied with blood. A variety of growth factors, and cartilage formation inducing agents, such as Cartilage Derived Morphogenetic Protein (CDMP), are also considered of value in this regard. Combinations of these elements is also desirable, and, no doubt, those skilled in the art, armed with the present disclosure, will be enabled to produce compositions of various descriptions which will be helpful in implementing this methodology.

Following implantation of the composition described above into or adjacent the necrotic site, the channel(s) is/are filled with a biologically compatible support member. While a wide variety of biologically compatible support members may be utilized for this purpose, it is considered preferable that a support member be chosen which will bioresorb, and even more preferable, remodel into bone, over the course of time. Any of a number of bioresorbable substances are known in the art, such as polyglycolic acid, polylactic acid and the like. In addition, a number of metallic screws may be useful for this purpose. Preferably, however, a screw made from bone, or a cylinder made from bone which exactly fits the channel created is utilized. Cortical bone interference screws have been utilized for the reconstruction of the anterior cruciate ligament, see U.S. Pat. No. 6,054,554, herein incorporated by reference. A bone screw adapted for the present purpose differs from the cortical bone screws used for ACL reconstruction in the '554 patent in that the bone screw of the present invention is preferably less tapered, or not tapered at all. In addition, the length of the screw for the present invention is preferably between about 25 mm to about 35 mm. Generally, a length of about 30 mm is acceptable for purposes of this invention. In addition, the diameter of the screw useful according the present invention is preferably between about 5 mm to about 10 mm, but most preferably is between about 7 mm and 8 mm in diameter. The screw may be composed completely of cortical bone, may be composed completely of cancellous bone, or may comprise both cortical bone and cancellous bone. In addition, the bone may be fully mineralized or demineralized. Demineralized bone screws are useful where rapid remodeling of the bone screw into the recipients bone is considered most desirable. In addition, the demineralized bone screw may be soaked or infused with growth factors, or bone progenitor cells, or both. A means for torquing the screw into a channel created according to this invention may be a slot on the head of the screw, or by use of a square driver head or any of the other torque means disclosed according to U.S. Pat. No. 6,054,554, which is incorporated herein by reference for this purpose. Threads may be inscribed over a portion or over the entire circumference of the screw.

Once the biologically compatible support member has been inserted into the channel(s) created to permit access to the site of necrosis, the support member also functions to maintain the osteogenic material (the term “osteogenic” is used herein to refer to osteoinductive substances, such as DBM, BMP, bone progenitor cells, and the like, as well as osteoconductive substances, such as bone chips, bioactive ceramics, and the like) within and adjacent to the site of necrosis. The surgery is completed by torquing the support member into place, and suturing the surgical site to permit healing to occur.

Having generally described the system, methodology, and compositions useful according to the present invention, the following specific embodiments are described to further describe and enable this invention. Those skilled in the art will appreciate, however, that while specific disclosure is provided with respect to a femoral head procedure, similar methodology may be applied at other sites in the musculoskeletal system with only minor modifications.

In one embodiment of the invention, biological and structural augmentation of a site in the hip affected by avascular necrosis is achieved by the following surgical technique. Those skilled in the art will appreciate that the specifics of this technique may be modified to some extent, without departing from the essence of the present invention.

1. With the patient positioned on a fracture table to permit image intensification during the procedure, the contralateral limb is maintained flexed at the hip and abducted.

2. The affected limb is positioned in extension and in sufficient internal rotation to neutralize the anteversion of the femoral neck.

3. The involved hip is then prepped and draped in a routine fashion.

4. Through a 5″ lateral midline incision, the vastus lateralis muscle is bluntly divided along the direction of its fibers in order to achieve subperiosteal exposure of the lateral aspect of the proximal femur.

5. Assuming 8 mm diameter implants, under image intensification a 9-millimeter entry hole is drilled.

6. A guide wire is inserted into the depth of the subchondral bone and a hole of diameter appropriate for an 8-millimeter tap is drilled nearly to the end of the guide wire.

7. Subsequent preparation of the canal is performed with an 8-millimeter reamer introduced to 75 to 80% of the intended length over the guide wire.

8. The remaining depth of the canal to the subchondral plate is completed with an 8-millimeter tap.

9. The guide wire is then removed and two to three 1 cc doses of demineralized bone matrix paste are introduced in the prepared canal using the prepackaged tuberculin syringes.

10. Under image intensification, an appropriate allograft or xenograft bone screw, for example an 8×30-millimeter allograft bone screw, is driven into the full depth of the canal.

11. Steps 6 to 8 are repeated to create a second diverging path and steps 9 andlo are completed in order to provide support to a sufficient subchondral area with the use of a second appropriate allograft or xenograft bone screw, such as an 8×30 millimeter bone screw.

12. The remainder of the canal is packed with a combination of cortico-cancellous bone chips and demineralized bone paste, or other appropriate material.

13. Routine irrigation and closure of the surgical wound.

14. The patient is kept on protected weight bearing for 6 weeks after the surgery.

It will be appreciated that variations in the specifics described above may be implemented. For example, alternatively, steps 6 and 7 are accomplished simultaneously by using a drill bit with stepped diameters. That is, a drill bit wherein the proximal 35 millimeters of the drill bit has a diameter appropriate for an 8-millimeter tap and the remainder of the shaft has an 8 mm diameter. In yet a further alternative, steps 5, 6, and 7 are accomplished simultaneously by using a drill bit with stepped diameters. That is a drill bit is utilized wherein the proximal 35 millimeters of the drill bit has a diameter appropriate for an 8 mm tap, the middle step has an 8 mm diameter and the remainder of the shaft has a 9 mm diameter. To accommodate different sizes of femur while minimizing the depth of the 9 mm diameter hole, the 9 mm diameter cutting edge is optionally in the form of an adjustable collar that is slid up or down the stepped 8 mm diameter drill bit.

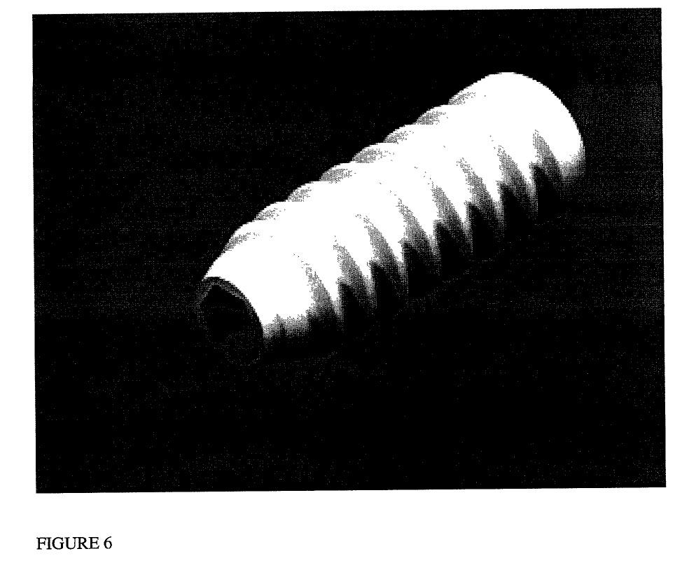

Referring to FIG. 6, there is shown a first embodiment of a screw made from cortical bone which is used according to this invention. In the embodiment shown in this figure, a diameter of between about 7 mm, 8 mm, or 9 mm is preferred. The length is perferably in the range of about 25-35 mm. Depending on the length of the channel, and the diameter, the screw length and diameter may be modified as necessary. The screw is preferably cannulated, preferably in the form of an hexagonal drive. Such form of cannulation is desirable as it spreads the torque for insertion of the screw over the entire length of the screw while providing excellent purchase for the driver device. As shown in this figure, the screw may be tapered at its front end, or it may be non-tapered. The decision to use a tapered or non-tapered screw is defined largely by the contours of the terminus of the channel that is formed into or adjacent the necrotic tissue. It is important that little if any void space remain in the channel once the screw is inserted. Thus, by matching a tapered channel terminus with a tapered screw, or a non-tapered channel terminus with a non-tapered screw, maximum support to the necrotic tissue is provided pending formation of new bone.

The present invention integrates several previously described treatment principles, and in so doing, meets a long-felt need for an improved method of treating or preventing osteonecrosis. The elevated intraosseous pressure is relieved with a standard core decompression which also improves vascularity and relieves pain [21] [20]. The resulting additional structural deficit associated with this procedure is addressed by the use of two bone screws. The bone healing process is accelerated and the incorporation of the bone screws is promoted through the use of osteoinductive demineralized bone allograft in the depth of the subchondral bone.

The long-term stabilization of the lesion is achieved with a reasonably simple procedure that involves a short postoperative period and low morbidity. It is intended that this multifactorial approach will result in resolution of the underlying orthopedic pathology and a reduction of the need for total hip replacement in these young patients. A prospective trial is being conducted to further assess the long-term benefits of this procedure.

Having generally and specifically described the invention claimed herein, a specific example is provided. Those skilled in the art will appreciate that the scope of this invention should not be perceived as restricted to the specifics of this example, but rather, by reference to the claims which follow.

Example of the Application of the Method of this Invention:

A 47 year/old white male with history of diabetes mellitus, hypercholesterolemia and avascular necrosis of his left hip for which he had a total hip replacement two years earlier, presents with right groin pain and painful range of motion.

An MRI study showed evidence of osteonecrosis of the right hip in pre-collapse stage (Ficat II) with a compromised area of 25% of the femoral head.

The patient underwent a biological and structural augmentation procedure as described herein. The surgery was well tolerated and there were no intraoperative complications. Postoperative films showed adequate placement of both bone screws. The patient was discharged from the Hospital the following day and was maintained on protected weight bearing for 6 weeks.

One month after surgery the patient was clinically asymptomatic exhibiting painless full range of motion of the hip. A follow-up x-ray study showed both screws in place with excellent bone response to the demineralized bone matrix material and cortico-cancellous chips allografts.

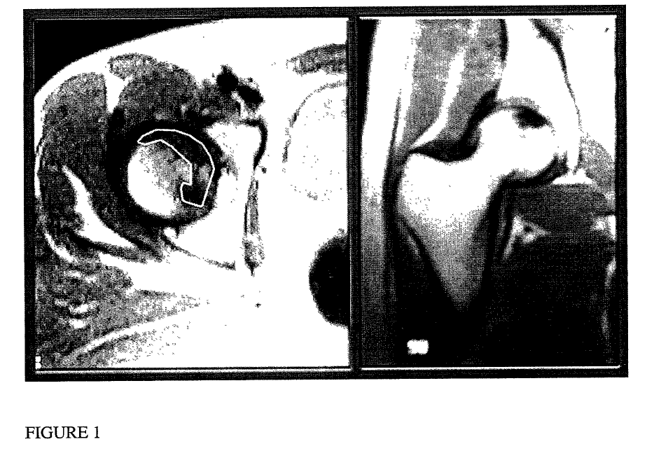

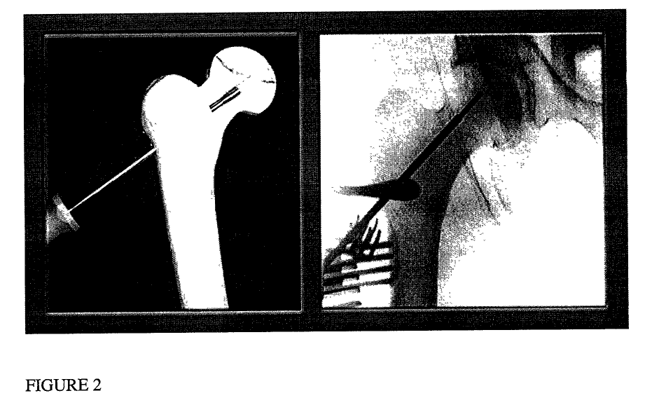

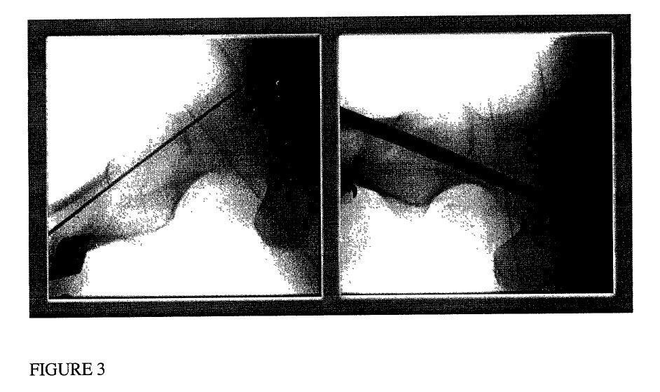

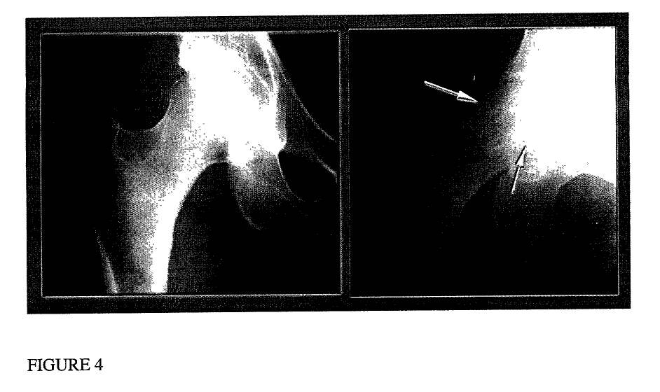

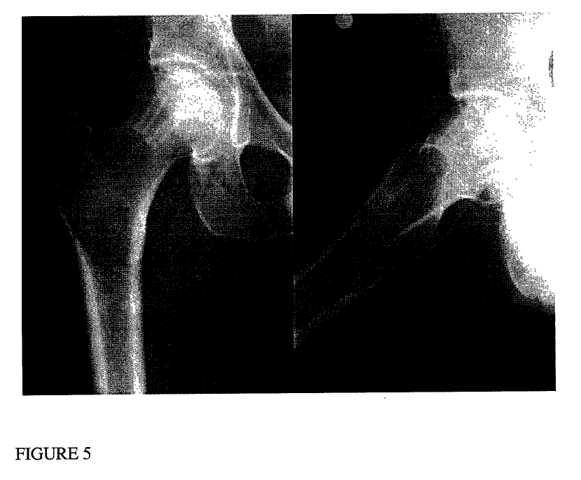

Follow-up MRI studies are conducted at 3 months after the initial procedure and at one year intervals as part of an ongoing prospective study and clinical follow-up. This series of surgical procedure and results is shown in FIGS. 1-5.

### REFERENCES

1. Mankin, H., Nontraumatic necrosis of bone (osteonecrosis). New England J. Med., 1992(326): p. 1473-1479.

2. Jacobs, B., Epidemiology of traumatic and nontraumatic osteonecrosis. Clin. Orthop, 1978. 130: p. 51-67.

3. Hasegawa, Y., et al., Vascularized pedicle bone-grafting for nontraumatic avascular necrosis of the femoral head. A 5-to 11-year follow-up. Arch Orthop Trauma Surg, 1997. 116(5): p. 251-8.

4. Chandler H P, R. F., Wixson R L, McCarthy J C, Total hip replacement in patients younger than thirty years old. A five-year follow-up study. J. Bone and Joint Surg., 1981. 63-A: p. 1426-1434.

5. Bobechko W P, H. W., The radiographic density of avascular bone. J Bone Joint Surg, 1960. 42 (B): p. 626.

6. Catto, M., Histological appearances of late segmental collapse of the femoral head after transcervical fracture. J Bone Joint Surg, 1965. 47 (B): p. 777.

7. Kenzora J E, S. R., Yosipovitch Z Y, et al, Tissue biology following experimental infarction of the femoral heads. J Bone Joint Surg, 1969. 51(A)(5).

8. Salter, R. B., Textbook of disorders and injuries of the musculoskeletal system. 1970, Baltimore: The William and Wilkins company.

9. Plancher K, R. A., Management of osteonecrosis of the femoral head. Orthopedic Clinics of North America, 1997. 28(3): p. 461-477.

10. Bonfiglio M, B. M., Treatment by bone grafting of aseptic necrosis of the femoral head and non-union of the femoral neck. J Bone Joint Surg, 1958. 40(A): p. 1329-1346.

11. Ohzono, K., et al., Natural history of nontraumatic avascular necrosis of the femoral head. J Bone Joint Surg Br, 1991. 73(1): p. 68-72.

12. Mont, M. A., J. J. Carbone, and A. C. Fairbank, Core decompression versus nonoperative management for osteonecrosis of the hip. Clin Orthop, 1996(324): p. 169-78.

13. Zangger, P., et al., Outcome of total hip replacement for avascular necrosis in systemic lupus erythematosus. J Rheumatol, 2000. 27(4): p. 919-23.

14. D'Antonio, J. A., et al., Hydroxyapatite coated implants. Total hip arthroplasty in the young patient and patients with avascular necrosis. Clin Orthop, 1997(344): p. 124-38.

15. Gonzalez, M. H., et al., Cementless total hip arthroplasty in patients with advanced avascular necrosis. J South Orthop Assoc, 1997. 6(3): p. 162-8.

16. Wei, S. Y., et al., Revision total hip arthroplasty in patients with avascular necrosis. Orthopedics, 1999. 22(8): p. 747-57.

17. Dorr, L. G., G. K. Takei, and J. P. Conaty, Total hip arthroplasty in patients less than forty five years old. J Bone Joint Surg, 1983. 65 A: p. 474-479.

18. Arlet, J. and P. Ficat, Forage-biopsie de la tete femorale dans l'osteonecrose primitive. Observations histo-pathologiques portant sur huitforages. Rev. Rhumat., 1964. 31: p. 257-264.

19. Arlet, J., Nontraumatic avascular necrosis of the femoral head. Past, present, and future. Clin Orthop, 1992(277): p. 12-21.

20. Schneider, W., et al., [The value of core decompression in treatment of femur head necrosis]. Orthopade, 2000. 29(5): p. 420-9.

21. Simank, H. G., et al., Long-term effects of core decompression by drilling. Demonstration of bone healing and vessel ingrowth in an animal study. Acta Anat, 1997. 158(3): p. 185-91.

22. Stulberg, B. N., et al., Osteonecrosis of the femoral head. A prospective randomized treatment protocol. Clin Orthop, 1991(268): p. 140-51.

23. Fairbank, A. C., et al., Long-term results of core decompression for ischaemic necrosis of the femoral head. J Bone Joint Surg Br, 1995. 77(1): p. 42-9.

24. Simank, H. G., et al., Comparison of results of core decompression and intertrochanteric osteotomy for nontraumatic osteonecrosis of the femoral head using Cox regression and survivorship analysis. J Arthroplasty, 2001. 16(6): p. 790-4.

25. Steinberg, M. E., et al., Core decompression with bone grafting for osteonecrosis of the femoral head. Clin Orthop, 2001(386): p. 71-8.

26. Yoon, T. R., et al., Failure after core decompression in osteonecrosis of the femoral head. Int Orthop, 2001. 24(6): p. 316-8.

27. Mazieres, B., et al., Influence of the volume of osteonecrosis on the outcome of core decompression of the femoral head. Ann Rheum Dis, 1997. 56(12): p. 747-50.

28. Bozic, K. J., D. Zurakowski, and T. S. Thornhill, Survivorship analysis of hips treated with core decompression for nontraumatic osteonecrosis of the femoral head. J Bone Joint Surg Am, 1999. 81(2): p. 200-9.

29. Chang, M. C., T. H. Chen, and W. H. Lo, Core decompression in treating ischemic necrosis of the femoral head. Chung Hua I Hsueh Tsa Chih (Taipei), 1997. 60(3): p. 130-6.

30. Boettcher, W. G., M. Bonfiglio, and K. Smith, Non-traumatic necrosis of the femoral head. II. Experiences in treatment. J Bone Joint Surg Am, 1970. 52(2): p. 322-9.

31. Buckley, P. D., P. F. Gearen, and R. W. Petty, Structural bone-grafting for early atraumatic avascular necrosis of the femoral head. J Bone Joint Surg Am, 1991. 73(9): p. 1357-64.

32. Springfield, D. S. and W. J. Enneking, Surgery for aseptic necrosis of the femoral head. Clin Orthop, 1978(130): p. 175-85.

33. Marcus, N. D., W. F. Enneking, and R. A. Massam, The silent hip in idiopathic aseptic necrosis. Treatment by bone-grafting. J Bone Joint Surg Am, 1973. 55(7): p. 1351-66.

34. Smith, K. R., M. Bonfiglio, and W. J. Montgomery, Non-traumatic necrosis of the femoral head treated with tibial bone-grafting. A follow-up note. J Bone Joint Surg Am, 1980. 62(5): p. 845-7.

35. Urbaniak, J. R. and E. J. Harvey, Revascularization of the femoral head in osteonecrosis. J Am Acad Orthop Surg, 1998. 6(1): p. 44-54.

36. Urbaniak, J. R., et al., Treatment of osteonecrosis of the femoral head with free vascularized fibular grafting. A long-term follow-up study of one hundred and three hips. J Bone Joint Surg Am, 1995. 77(5): p. 681-94.

37. Brunelli, G., et al., Free microvascular fibular versus conventional bone grafts. Int Surg, 1991. 76(1): p. 33-42.

38. Zhu, S. X. and M. W. Zhou, Iliac periosteal graft with vascular pedicle in the treatment of avascular necrosis of the femoral head. Experimental study and clinical application. Chin Med J (Engl), 1992. 105(10): p. 849-55.

39. Scully, S. P., R. K. Aaron, and J. R. Urbaniak, Survival analysis of hips treated with core decompression or vascularized fibular grafting because of avascular necrosis. J Bone Joint Surg Am, 1998. 80(9): p. 1270-5.

40. Soucacos, P. N., et al., Treatment of avascular necrosis of the femoral head with vascularized fibular transplant. Clin Orthop, 2001(386): p.120-30.

41. Vail, T. P. and J. R. Urbaniak, Donor-site morbidity with use of vascularized autogenous fibular grafts. J Bone Joint Surg Am, 1996. 78(2): p. 204-11.

42. Aluisio, F. V. and J. R. Urbaniak, Proximal femur fractures after free vascularized fibular grafting to the hip. Clin Orthop, 1998(356): p. 192-201.

What is claimed is:

1. A system for treating and/or preventing the complications associated with avascular necrosis in the musculoskeletal system of a human or non-human patient which comprises:
a. identifying the biological site in the musculoskeletal system of said patient in need of said treatment;
b. creating at least one channel from the exterior of said biological site in said musculoskeletal system into or distal to said site in a manner that facilitates access to said site; and
c. inserting into said at least one channel a combination comprising at least (i) a composition comprising an osteoinductive element, an osteoconductive element or both and (ii) a biologically compatible support member which substantially fills said channel and which provides support to the biological site in the musculoskeletal system of said patient pending formation of new bone and vasculature at said site.

2. The system according to claim 1 wherein said composition comprising said osteoinductive element, said osteoconductive element or both, further comprises at least one angiogenic element.

3. The system according to claim 1 wherein said osteoinductive element comprises demineralized bone matrix (DBM), bone morphogenetic protein (BMP), cartilage derived morphogenetic protein (CDMP), bone progenitor cells, a growth factor, or combinations thereof.

4. The system according to claim 1 wherein said osteoconductive element comprises cortical bone chips, cancellous bone chips, chips which have both a cortical and a cancellous nature, mixtures of cortical bone chips and cancellous bone chips, bioactive ceramic, a calcium salt composition, a phosphate salt composition, or combinations thereof.

5. The system according to claim 1 wherein said composition comprises a biologically compatible carrier matrix.

6. The system according to claim 5 wherein said biologically compatible carrier matrix comprises gelatin, hyaluronic acid, glyscosaminoglycan, glycerol, alginate, methacrylate, methyl-methacrylate, or combinations thereof.

7. The system according to claim 1 wherein said site exhibiting avascular necrosis in the musculoskeletal system is located within the head of the femur, in the femoral or tibial condyles or epicondyles, the malleolus of the tibia or fibula, the head or tubercles of the humerus, or either terminus of the radius or ulna, talus, carpal navicular.

8. The system according to claim 1 wherein said biologically compatible support member which substantially fills said channel and which provides support to the biological site in the musculoskeletal system of said patient pending formation of new bone and vasculature at said site comprises a biologically compatible synthetic material, a biologically compatible metal, a cortical shaft of bone, a cancellous shaft of bone, a shaft of bone that comprises both cortical bone and cancellous bone.

9. The system according to claim 8 wherein said biologically compatible support member is in the form of a plug that substantially fills said channel.

10. The system according to claim 9 wherein said plug comprises threading over at least a portion of its circumference such that said plug maybe torqued into place within said channel.

11. The system according to claim 10 wherein said plug comprising threading over at least a portion of its circumference further comprises a means for engagement with a torque delivery mechanism.

12. A kit for treating avascular necrosis in the musculoskeletal system of a human or non-human patient wherein said treating comprises:
a. identifying the biological site in the musculoskeletal system of said patient in need of said treatment;
b. creating at least one channel from the exterior of said biological site in said musculoskeletal system distal or into said site in a manner that facilitates access to said site; and
c. inserting into said at least one channel a combination comprising at least (i) a composition comprising an osteoinductive element, an osteoconductive element or both and (ii) a biologically compatible support member which substantially fills said channel and which provides support to the biological site in the musculoskeletal system of said patient pending formation of new bone and vasculature at said site;
wherein said kit comprises:
i. at least one sterile or sterilizable drill bit for creating said channel;
ii. at least one composition comprising said osteoinductive element, said osteoconductive element or both;
iii. at least one biologically compatible support member; and
iv. at least one device adapted for insertion of said biologically compatible support member into said channel.

13. The kit according to claim 12 wherein said drill bit is cannulated, and has a diameter of between about 5 mm and about 10 mm.

14. The kit according to claim 12 wherein said drill bit comprises at least one steps in its diameter, including in its cutting edge and optionally including an adjustable collar that may be slid axially along the drill bit.

15. The kit according to claim 12 further comprising a tap for inscribing threads within said channel, wherein said tap is cannulated, and has a diameter of between about 5 mm and about 10 mm.

16. The kit according to claim 12 further comprising at least one guidewire.

17. The kit according to claim 12 wherein said device adapted for insertion of said biologically compatible support member into said channel is cannulated

18. The kit according to claim 12 further comprising a delivery device for inserting into said at least one channel said composition comprising an osteoinductive element, an osteoconductive element or both.

19. The kit according to claim 18 wherein said delivery device comprises a tamp, a syringe, or both.

20. The kit according to claim 12 wherein said biologically compatible support member which substantially fills said channel and which provides support to the biological site in the musculoskeletal system of said patient pending formation of new bone and vasculature at said site comprises a biologically compatible synthetic material, a biologically compatible metal, a cortical shaft of bone, a cancellous shaft of bone, a shaft of bone that comprises both cortical bone and cancellous bone.

21. The kit according to claim 20 wherein said biologically compatible support member is in the form of a plug that substantially fills said channel.

22. The kit according to claim 21 wherein said plug comprises threading over at least a portion of its circumference such that said plug may be torqued into place within said channel.

23. The kit according to claim 22 wherein said plug comprising threading over at least a portion of its circumference further comprises a means for engagement with a torque delivery mechanism.

24. The kit according to claim 20 wherein said biologically compatible support member comprises substantially cortical bone.

25. The kit according to claim 24 wherein said substantially cortical bone support member is cannulated.

26. The kit according to claim 24 wherein said substantially cortical bone support member is of a length of about 25 mm to about 35 mm.

27. The kit according to claim 26 wherein said substantially cortical bone support member is at least partially demineralized.

28. The kit according to claim 12 further comprising an instrument tray.

29. A screw made from bone comprising a cylinder of bone, with or without taper, of a length between about 25 mm and about 35 mm, and a width of between about 5 mm and about 10 mm.

30. The screw made from bone according to claim 29 wherein said screw comprises a thread inscribed in the circumference of the screw over at least a portion of the circumference thereof.

31. The screw made from bone according to claim 29 wherein said screw is cannulated.

32. The screw made from bone according to claim 29 wherein said bone is partially or completely demineralized.

33. The screw according to claim 32 wherein said screw has been soaked or infused with growth factors, BMP, bone progenitor cells, or combinations thereof.

34. The screw according to claim 29 comprising cortical bone, cancellous bone, or both cortical and cancellous bone.

35. A method of treating or preventing avascular necrosis in the femur of a human or non-human patient which comprises:
a. Appropriately positioning the patient to provide access to the affected limb;
b. Forming an incision to permit exposure of the aspect of the femur affected by the avascular necrosis;
c. Creating an entry hole in the femur;
d. Inserting at least one guidewire into the femur directed toward the necrotic site and utilizing said guidewire as a guide to create at least one channel directed toward the site of necrosis, which channel may be tapped;
e. Removing said guidewire and introducing into said channel a bone-inducing amount of an osteoinductive substance;
f. Inserting into said channel at least one biocompatible support member; and
g. Closing said incision to permit healing.

36. The method according to claim 35 comprising:
a. Positioning said patient on a fracture table to permit image intensification during the procedure, with the contralateral limb maintained flexed at the hip and abducted;
b. Positioning the affected limb in extension and in sufficient internal rotation to neutralize the anteversion of the femoral neck;
c. Prepping and draping the involved hip for surgery;
d. Forming an approximately 5″ lateral midline incision, dividing the vastus lateralis muscle bluntly along the direction of its fibers in order to achieve subperiosteal exposure of the lateral aspect of the proximal femur;
e. Drilling, under image intensification, an entry hole of between about 7 to 10 millimeters in diameter;
f. Inserting a guide wire into the depth of the subchondral bone;
g. Preparing a canal or channel into the bone with a reamer of about 9 millimeters in diameter introduced to about 80 to 90% of the intended length over the guide wire;
h. Forming the remaining depth of the canal or channel to the subchondral plate with a tap of an about 8 millimeter diameter;
i. Removing the guide wire;
j. Introducing into the canal or channel two to three cubic centimeters of an osteoinductive composition selected from the group consisting of demineralized bone matrix, bone morphogenetic protein, angiogenic factors, cartilage derived growth factor, and combinations thereof;
k. Driving, under image intensification, an allograft or xenograft bone screw into the full depth of the canal;
l. Repeating steps f-k to create a second diverging path and to provide support to a sufficient subchondral area with the use of a second allograft or xenograft bone screw;
m. Packing the remainder of the canal with an osteoconductive or an osteoinductive composition selected from the group consisting of bone chips, demineralized bone matrix, bone morphogenetic protein, angiogenic factors, cartilage derived growth factor, and combinations thereof;
n. Irrigating and closing the surgical wound; and
o. Maintaining the patient on protected weight bearing for approximately 6 weeks after the surgery.
